(19) United States
(12) Patent Application Publication
(10) Pub. No.: US 2006/0211670 A1
(43) Pub. Date: Sep. 21, 2006

(54) Antitumoral18a-homoestra-1,3,5(10)-trien-3yl 2-substituted sulfamates

(76) Inventors: Alexander Hillisch, Velbert (DE); Olaf Peters, Tabarz (DE); Christian Gege, Ehingen (DE); Gerhard Siemeister, Berlin (DE); Eberhard Unger, Cospeda (DE); Ina Scherlitz-Hofmann, Falkensee (DE); Wilko Regenhardt, Osterode (DE)

Correspondence Address: MILLEN, WHITE, ZELANO & BRANIGAN, PC, 2200 CLARENDON BLVD, SUITE 1400, ARLINGTON, VA, 22201, US

(21) Appl. No.: 10/546,025

(22) PCT Filed: Feb. 19, 2004
(86) PCT No.: PCT/EP04/01630

§ 371 Date: Aug. 18, 2005

(30) Foreign Application Priority Data
Feb. 19, 2003 (EP) 103 07 105.9

(51) Int. Cl. A61K 31/56; C07J 1/00

(52) U.S. Cl. 514/182; 552/626

(57) ABSTRACT

The invention relates to the use of compounds of a general formula (I), wherein R², R¹⁴ to R¹⁷ and X groups have the following significance: R2 is C1-C5-Alkyl, C1-C5-Alkoxy or a —O—C<SB>N</SB>FₘHₒ rest, wherein n=1,2,3,4,5 or 6, m>1 and m+o=2n+1, R14 and R15 separately designate hydrogen or together a methyl group or a supplementary liaison, R16 is hydrogen; R17 is hydrogen, hydroxy, C1-C5-Alkyl, C1-C5-Alkoxy or SO₃NHX; X is hydrogen, C1-C5-alkyl or C1-C5 acyl. In the cycle B and D of a steroid skeleton, stippled lines can additionally represent up to two double bonds. The salts of said compounds which are pharmaceutically acceptable for preparing a drug are also disclosed.

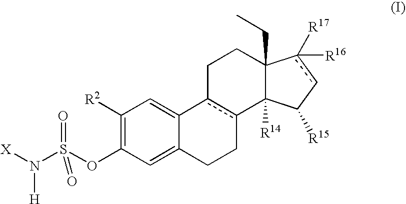

This invention relates to 2-substituted 18a-homoestra-1,3,5(10)-trien-3-yl sulfamates and use thereof for the production of pharmaceutical agents that have an antitumor-active activity.

Microtubuli are organelles that occur in most eukaryotic cells and take over a number of functions there such as mitosis, intracellular movements, cell migration and the manifestation of the cell shape. Microtubuli are polymers that consist of tubulin, which in turn represents a dimer that consists of an α-unit and a β-unit. These heterodimers bind two guanosine triphosphate (GTP) molecules, whereby one of the GTPs is securely bonded and the other is replaceable. In a head-tail arrangement, the heterodimers polymerize into thread-shaped macromolecules, the so-called protofilaments, which in turn pile up into tubular organelles, the microtubuli. Microtubuli are subject to a constant build-up and degradation. The equilibrium between growth and degradation depends on the availability of new GTP-tubulin subunits and the rate of hydrolysis of the second bonded GTPs. On the plus end, new subunits are cultivated; conversely, on the minus end, subunits diffuse outward. It is known that cytotoxic substances such as colchicine, vinblastine, vincristine, taxol, epothilone, podophyllotoxin, steganicin, combretastatin and methoxyestradiol influence the build-up or degradation of mictrotubuli (tubulin polymerization and tubulin depolymerization) and thus are able to influence the cell division in a phase-specific manner. This relates primarily to quick-growing, neoplastic cells, whose growth is largely unaffected by intracellular regulating mechanisms. Active ingredients of this type are in principle suitable for treating malignant tumors.

Fotsis et al. Nature 1994 368, 237-239 report, moreover, that 2-methoxyestradiol inhibits the tumor growth and the angiogenesis.

Cushman et al. J. Med. Chem. 1995 38, 2041-2049 examine the cytotoxic action as well as the tubulin-polymerization-inhibiting action of 2-methoxyestradiol and report in J. Med. Chem. 1997, 40, 2323-2334, moreover, that 2-alkoxy-6-oximinoestradiol derivatives inhibit the tubulin polymerization as well as the bond of [³H]-colchicine to tubulin. The 2-alkoxy-6-oximinoestradiol derivatives that are mentioned here show comparable activity, relative to the inhibition of tubulin polymerization, such as 2-ethoxyestradiol, which has a higher activity than 2-methoxyestradiol.

WO 93/05064 relates to, i.a., compounds of formula 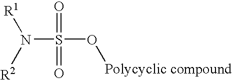
whereby R¹ and R², in each case independently of one another, mean hydrogen or a methyl group, provided that at least one of radicals R¹ and R² is an H atom, and the radical-O-polycyclic compound is a 3-sterol, whose sulfate ester can be hydrolyzed by an enzyme with steroid-sulfatase activity. Compounds that are substituted specifically in the 2-position of the steroid skeleton are not explicitly disclosed.

U.S. Pat. No. 6,011,024 is based on WO 93/05064 and covers, e.g., all compounds in which the primary sulfamate function is bonded to a six-membered ring. Compounds that are specifically substituted in the 2-position of the steroid skeleton are in turn not explicitly disclosed.

WO 96/05216 relates to C2-unsubstituted estra-1,3,5(10)-triene-sulfamate derivatives.

WO 96/05217 relates to pharmaceutical compositions that contain active ingredients of general formula 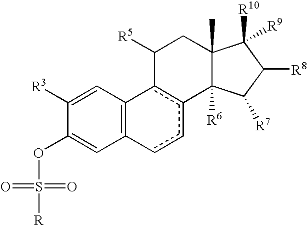
in which R═NH₂; R³=C₁₋₅-alkoxy group, OH; R⁸, R⁹ and R¹⁰, independently of one another, ═H, OH; R⁹ and R¹⁰ together can have the meaning=0. The pharmaceutical compositions that are disclosed therein can be used for female birth control; menopausal HRT and for treatment of gynecological and andrological images of disease, such as breast cancer or prostate cancer.

WO 97/14712 relates to steroid sulfamate derivatives of general formula 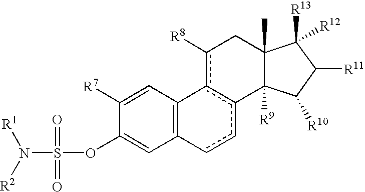
in which R¹ can represent an acyl, alkoxycarbonyl, aminocarbonyl, sulfonyl or sulfonamidyl group; R² can represent a hydrogen atom or a metal atom; R⁷ and R⁸, independently of one another, can represent H, OH and C₁₋₅-alkoxy; R¹³, R¹² and R¹¹, independently of one another, can represent H or OH.

WO 98/42729 relates to 16-halogen-substituted 1,3,5-estratriene-3-monosulfamates as well as 3,17β-bissulfamates, which can be alkoxy-substituted at C2. The 16-halogen substitution increases both the sulfatase-inhibiting action and the estrogeneity of the corresponding sulfamate derivatives.

The introduction of a 17-sulfamate function in addition to the 3-sulfamate function drastically reduces the estrogeneity.

WO 98/24802 relates to sulfamates that inhibit the estrone sulfatase. 2-Methoxyestrone sulfamate is explicitly mentioned. As a potential therapeutic application, breast cancer, but not prostate cancer, is mentioned in the description.

Also, WO 99/33858 describes estrone sulfatase inhibitors of formula 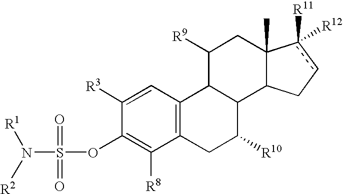
in which R¹ and R², independently of one another, represent H, alkyl, or together piperidine, morpholine, piperazine; R³═H, CN, NO₂, CO₂R⁴; R⁸═H, NO₂, NR⁶R⁷. In the description, breast cancer is mentioned as a possible therapeutic application.

WO 99/64013 relates to a pharmaceutical composition of a sulfamate derivative with a cell signal modifier (such as, e.g. TNFα). 2-Methoxyestrone sulfamate is explicitly claimed as a preferred sulfamate in this combination; but numerous other steroid-3-sulfamates fall under the scope of the general formula. As a mechanism of action for the pharmaceutical compositions according to the invention or for the steroid-3-sulfamates contained therein (preferably with at least one 2-alkoxy substituent), 1) inhibition of the glucose absorption in tumor cells, 2) inhibition of tumor angiogenesis, 3) degradation of microtubuli; 4) inducing of apoptosis are described. WO 00/76487 relates to substances that inhibit the TNFα-induced aromatase activity. As such, 2-alkoxyestrone-3-sulfamates, preferably 2-methoxyestrone sulfamate, are claimed.

WO 01/18028 describes non-estrogenic estrone sulfatase-inhibiting N-acyl-18a-substituted steroid-3-sulfamates, such as, e.g., 16α-fluoro-2-methoxy-18a-homoestradiol-(N-acetylsulfamate) or 16α-fluoro-2-methoxy-18a-homoestrone-(N-acetylsulfamate).

In Cancer 2000, 85, 983-994, the 2-methoxyestradiol, docetaxel and paclitaxel-induced apoptosis in hepatoma cells and their correlation with reactive oxygen species are compared.

Potter et al. Int. J. Cancer 2000, 85, 584-589 examine the action of 2-methoxyestrone sulfamate in comparison to 2-methoxyestrone on the growth of breast cancer cells and induced breast tumors and find that 2-methoxyestrone sulfamate has a significant therapeutic potential for treating breast cancer.

Potter et al. Molecular and Cellular Endocrinology 2000, 160, 61-66 examine the inhibition of deoxyglucose absorption in MCF-7 breast cancer cells by 2-methoxyestrone and 2-methoxyestrone-3-sulfamate, which inhibit glucose absorption by 25 to 49% with 10 μm (also 2-methoxyestradiol and 2-methoxyestrone), and it follows that the compounds could have therapeutic potential for inhibiting breast cancer by their capacity to inhibit glucose absorption.

Potter et al. Cancer Research 2000, 60, 5441-5450 describe 2-methoxyestrone-sulfamate and 2-ethoxyestrone sulfamate as new antimicrotubulin-active compounds that have in-vitro anti-cancer activity in breast cancer cells and therefore also optionally could be active in vivo. In J. Steroid Biochem. and Mol. Biol. 1999, 69, 227-238, it is reported that the inhibition of the steroid sulfatase activity is an important starting point in the treatment of hormone-dependent breast cancer. 2-Methoxyestrone sulfamate, 17-deoxyestrone sulfamate and estrone sulfamate are cited explicitly. Monocyclic or bicyclic, non-steroidal sulfamates namely inhibit the steroid sulfatase, but not as effectively as the corresponding steroid derivatives.

The object of this invention consists in making available additional compounds that effectively inhibit the tubulin polymerization.

The object of this invention is achieved according to the invention by the use of 2-substituted 18a-homoestra-1,3,5(10)-trien-3-yl sulfamates of general formula I for the production of a pharmaceutical agent, in particular for treating tumor diseases, which can be influenced positively by the inhibition of tubulin polymerization: 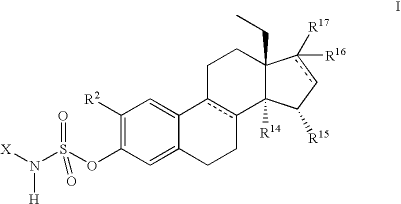
in which
R² means C₁-C₅-alkyl, C₁-C₅-alkoxy or a radical —O—CₙFₘHₒ, whereby n=1, 2, 3, 4, 5 or 6, m>1, and m+o=2n+1,
R¹⁴ and R¹⁵ in each case mean hydrogen or together a methylene group or an additional bond,
R¹⁶ means hydrogen,
R¹⁷ means hydrogen, hydroxy, C₁-C₅-alkyl, or C₁-C₅-alkoxy or a radical —CₙFₘHₒ,
   whereby n=1, 2, 3, 4, 5, or 6, m>1 and m+o=2n+I
   or a group SO₃NHX,
X means hydrogen, C₁-C₅-alkyl or C₁-C₅-acyl,
   whereby in the B- and D-ring of the steroid skeleton, the dotted lines can also be up to two double bonds,
   as well as their pharmaceutically acceptable salts.

It was determined that the 2-substituted 18a-homoestra-1,3,5(10)trien-3-yl sulfamates according to the invention more greatly inhibit in vitro the tubulin polymerization, surprisingly enough, than 2-methoxyestradiol itself. The compounds according to the invention inhibit the proliferation of tumor cells and also show in-vivo antitumor action. Moreover, the above-mentioned compounds have good oral bioavailability.

Alkyl radicals are defined as straight-chain or branched-chain, saturated or unsaturated alkyl radicals. As representatives of straight-chain or branched-chain alkyl groups with 1-5 carbon atoms, for example, methyl, ethyl, propyl, isopropyl, butyl, isobutyl, tert-butyl, pentyl, 1-ethylpropyl, 1-methylbutyl, 1,1-dimethylpropyl, 2-methylbutyl, 1,2-dimethylpropyl, 3-methylbutyl, 2,2-dimethylpropyl can be mentioned.

For example, allyl, vinyl, propenyl, butenyl, but also ethinyl, propinyl or butinyl stand for unsaturated alkyl radicals.

Acyl radicals mean, for example, formyl, acetyl, propionyl, butyryl, iso-butyryl or valeryl.

A methoxy, ethoxy, n-propoxy, iso-propoxy, n-butoxy, iso-butoxy, tert-butoxy or pentoxy group can stand for a C₁-C₅-alkoxy radical, whereby the latter can be fluorinated.

Preferred according to this invention is the use of those compounds of general formula I, in which:
R² means methyl, ethyl, methoxy, ethoxy, or 2,2,2-trifluoroethoxy, R¹⁴ and R¹⁵ in each case mean H, or together a methylene group,
R¹⁶ means hydrogen,
R¹⁷ means hydrogen, hydroxy, OC₁-C₅-alkyl or a radical —CₙFₘHₒ,
   whereby n=1, 2, 3, 4, 5 or 6, m>1 and m+o=2n+1
   or a group SO₃NHX,
X means hydrogen, methyl, acetyl, propionyl or butyryl,
whereby in the B- and C-ring of the steroid skeleton, the dotted lines can mean an 8,9-double bond or a 16,17-double bond.

The use of the compounds mentioned below is especially preferred:
1. 17β-Hydroxy-2-methoxy-18a-homoestra-1,3,5(10),14-tetraen-3-yl sulfamate
2. 17α-Hydroxy-2-methoxy-18a-homoestra-1,3,5(10),14-tetraen-3-yl sulfamate
3. 2,17β-Dimethoxy-18a-homoestra-1,3,5(10),14-tetraen-3-yl sulfamate
4. 2,17α-Dimethoxy-18a-homoestra-1,3,5(10),14-tetraen-3-yl sulfamate
5. 2-Methoxy-18a-homoestra-1,3,5(10),14-tetraen-3-yl sulfamate
6. 17β-Hydroxy-2-methoxy-18a-homoestra-1,3,5(10)trien-3-yl sulfamate (1)
7. 17β-Hydroxy-2-methoxy-18a-homoestra-1,3,5(10)trien-3-yl (N-acetyl)-sulfamate
8. 17α-Hydroxy-2-methoxy-18a-homoestra-1,3,5(10)trien-3-yl sulfamate
9. 2-Ethoxy-17β-hydroxy-18a-homoestra-1,3,5(10)-trien-3-yl sulfamate (2)
10. 2-Ethoxy-17β-hydroxy-18a-homoestra-1,3,5(10)-trien-3-yl (N-acetyl)-sulfamate
11. 2-Ethoxy-17α-hydroxy-18a-homoestra-1,3,5(10)-trien-3-yl sulfamate
12. 2,17β-Dimethoxy-18a-homoestra-1,3,5(10)-trien-3-yl sulfamate (5)
13. 17β-Ethoxy-2-methoxy-18a-homoestra-1,3,5(10)-trien-3-yl sulfamate
14. 2-Methoxy-17β-(1-propyloxy)-18a-homoestra-1,3,5(10)-trien-3-yl sulfamate
15. 2-Methoxy-18a-homoestra-1,3,5(10)-trien-3-yl sulfamate (4)
16. 2-Methoxy-18a-homoestra-1,3,5(10)-trien-3-yl (N-acetyl)-sulfamate
17. 2-Methoxy-18a-homoestra-1,3,5(10),16-tetraen-3-yl sulfamate
18. 2-Methoxy-18a-homoestra-1,3,5(10),16-tetraen-3-yl (N-acetyl)-sulfamate
19. 2-Ethyl-17β-methoxy-18a-homoestra-1,3,5(10)-trien-3-yl sulfamate
20. 17β-Ethoxy-2-ethyl-18a-homoestra-1,3,5(10)-trien-3-yl sulfamate
21. 2-Methoxy-18a-homoestra-1,3,5(10)-triene-3,17β-diyl bissulfamate
22. 2-Methoxy-18a-homoestra-1,3,5(10)-triene-3,17β-diyl bis-(N-acetyl) sulfamate (3)
23. 2-Ethoxy-18a-homoestra-1,3,5(10)triene-3,17β-diyl bissulfamate (3)
24. 2-Ethoxy-18a-homoestra-1,3,5(10)triene-3,17β-diyl bis-(N-acetyl)sulfamate
25. 2-Ethyl-18a-homoestra-1,3,5(10)-triene-3,17β-diyl bissulfamate (6)
26. 2-Ethyl-18a-homoestra-1,3,5(10)-triene-3,17β-diyl bis-(N-acetyl)-sulfamate

Subjects of this invention are, moreover, compounds of general formula I 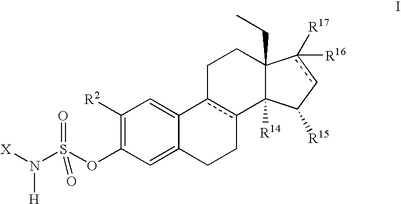
in which
R² means C₁-C₅-alkyl, C₁-C₅-alkoxy or a radical —O—CₙFₘHₒ, whereby n=1, 2, 3, 4, 5 or 6, m>1 and m+o=2n+1,
R¹⁴ and R¹⁵, in each case mean hydrogen or together a methylene group,
R¹⁶ means hydrogen,
R¹⁷ means hydrogen, hydroxy, C₁-C₅-alkyl or C₁-C₅-alkoxy, or a radical —CₙFₘHₒ,
   whereby n=1, 2, 3, 4, 5 or 6, m>1 and m+o=2n+1,
X means hydrogen, C₁-C₅-alkyl,
   whereby in the B- and D-ring of the steroid skeleton the dotted lines can also be up to two double bonds,
   as well as their pharmaceutically acceptable salts.

Alkyl radicals, unsaturated alkyl radicals and C₁-C₅-alkoxy radicals, which can be fluorinated, are defined as the same radicals as already embodied in the use according to the invention.

Preferred are 2-substituted 18a-homoestra-1,3,5(10)-trien-3-yl sulfamates of general formula I, in which:
R² represents methyl, ethyl, methoxy, ethoxy or 2,2,2-trifluoroethoxy,
R¹⁴ and R¹⁵ in each case represent H or together a methylene group,
R¹⁶ represents hydrogen,
R¹⁷ represents hydrogen, hydroxy or OC₁-C₅-alkyl,
X represents hydrogen, methyl,
   whereby in the B- and C-ring of the steroid skeleton, the dotted lines can mean an 8,9-double bond or a 16,17-double bond.

The compounds that are mentioned below are especially preferred according to the invention:
1. 17β-Hydroxy-2-methoxy-18a-homoestra-1,3,5(10)-trien-3-yl sulfamate
2. 17α-Hydroxy-2-methoxy-18a-homoestra-1,3,5(10)-trien-3-yl sulfamate
3. 2-Ethoxy-17β-hydroxy-18a-homoestra-1,3,5(10)-trien-3-yl sulfamate
4. 2-Ethoxy-17α-hydroxy-18a-homoestra-1,3,5(10)-trien-3-yl sulfamate
5. 2,17β-Dimethoxy-18a-homoestra-1,3,5(10)-trien-3-yl sulfamate
6. 17β-Ethoxy-2-methoxy-18a-homoestra-1,3,5(10)-trien-3-yl sulfamate
7. 2-Methoxy-17β-(1-propyloxy)-18a-homoestra-1,3,5(10)-trien-3-yl sulfamate
8. 2-Methoxy-18a-homoestra-1,3,5(10)-trien-3-yl sulfamate
9. 2-Methoxy-18a-homoestra-1,3,5(10),16-tetraen-3-yl sulfamate
10. 2-Ethyl-17β-methoxy-18a-homoestra-1,3,5(10)-trien-3-yl sulfamate
11. 17β-Ethoxy-2-ethyl-18a-homoestra-1,3,5(10)-trien-3-yl sulfamate
   Pharmacological Data
   1. Inhibition of Tubulin Polymerization

The compounds according to the invention were tested in various models.

The compounds of general formula I according to the invention are distinguished in that they more greatly inhibit tubulin polymerization than 2-methoxyestradiol. The in-vitro testing of the tubulin polymerization influence was performed as follows:

According to Shelanski et al. (Shelanski et al. Proc. Natl. Acad. Sci. USA 1973, 70, 765-8), microtubular protein was purified from pig brains via cydic assembling/disassembling. The buffer system used had the following composition: 20 mmol of PIPES (1,4-piperazine-diethane-sulfonic acid, pKa 6.8), 80 mmol of NaCl, 0.5 mmol of MgCl₂, 1 mmol of EGTA (ethylene glycol-bis-(2-aminoethylene)-tetraacetic acid).

For active ingredient testing, protein concentrations of 1 mg/ml (about 10⁻⁵ mmol of tubulin) were used. The determination of protein was carried out according to the Lowry Method (Lowry et al. J. Biol. Chem. 1951, 193, 265-75) with bovine serum albumin as a standard. The assembling of microtubuli was carried out in the presence of 0.25 mmol of GTP and heating the samples to 37° C.

The microtubulus formation was examined by means of turbidimetry at a wavelength of 340 nm. The state of equilibrium, in which the microtubular protein exhibits no increase in the assemblate concentration (corresponding to the microtubulus concentration) and the turbidity value no longer exhibits an increase, is typically reached after 20 minutes.

Testing of the active ingredients was carried out by their addition at the beginning of the assembling or in the state of equilibrium. Deviations of turbidity curves from the control characterize its activity. To monitor action and to evaluate the measured turbidity values, a transmission electron microscopic study (CEM 902 A, Zeiss/Oberkochen) of the assemblates was always performed after negative staining with 1% aqueous uranyl acetate.

TABLE 1

| | Inhibition of Tubulin Polymerization |
|---|---|
| Compound | IC₅₀ [μm] |
| 2-Methoxyestradiol | 2.70 |
| (1) | 0.50 |
| (4) | 1.80 |

2. Inhibition of Cell Proliferation

The compounds according to the invention are distinguished by a potent inhibition of cell proliferation.

Cell cultures of the following cell lines were prepared in 96-well microtiter plates:
1. MaTu/ADR multidrug-resistant human breast tumor cells (Epo GmbH Berlin), 5000 cells/well.
2. HCT116 human colon tumor cells (ATCC CCL-247), 3000 cells/well.
3. NCl-H460 human non-small-cell lung cancer cells (ATCC HTB-177), 3000 cells/well.
4. DU145 human prostate tumor cells (ATCC HTB-81), 5000 cells/well.
5. HMVEC human primary dermal microvascular endothelial cells, 7500 cells/well.

After 24 hours of incubation in a cell culture incubator at 37° C., the cells of a microtiter plate were stained with crystal violet (reference plate), while the cells in the test plates were incubated for 4 days with the test substances in the concentrations 0.1-10 μm, as well as with the DMSO solvent by itself (solvent control). The cell proliferation was determined by staining cells with crystal violet. The extinction of the crystal violet was determined by photometry at 595 nm. The percentage of the change in the cell number in the test plates was determined after the extinction values were normalized to the reference plate (0%) and to the solvent control (100%). The semi-maximal inhibition of the cell growth (IC50) was determined as the substance concentration, in which 50% of the cell number of the solvent controls were present.

TABLE 2

| | Inhibition of Cell Proliferation IC50 [μm] | | | | |
|---|---|---|---|---|---|
| | | | | MaTu/ | |
| Compound | NCl-H460 | HCT116 | DU145 | ADR | HMVEC |
| Taxol | 0.004 | 0.004 | 0.004 | 0.4 | 0.004 |
| 2- | 1.8 | 1.1 | 1.9 | 0.2 | 2.2 |
| Methoxyestradiol | | | | | |
| (4) | 0.5 | 0.6 | 0.6 | 0.45 | 0.6 |
| (1) | <0.1 | <0.1 | 0.15 | <0.1 | <0.1 |
| (2) | 0.3 | 0.4 | 0.9 | 0.1 | 0.5 |
| (5) | 0.13 | <0.1 | 0.19 | <0.1 | <0.1 |
| (6) | 0.18 | 0.18 | 0.18 | 0.1 | <0.1 |

Dosage

In general, satisfactory results can be expected when the daily doses comprise a range of 5 μg to 50 mg of the compound according to the invention per kg of body weight. In larger mammals, for example in humans, a recommended daily dose is in the range of 10 μg to 30 mg per kg of body weight.

Suitable dosages for the compounds according to the invention are from 0.005 to 50 mg per day per kg of body weight, depending on the age and constitution of the patient, whereby the necessary daily dose can be administered one or more times.

Based on the special depot action of the estrogen-sulfamates, the compounds according to the invention can, however, also be administered at greater intervals than once per day.

The formulation of the pharmaceutical preparations based on the new compounds is carried out in a way that is known in the art, by the active ingredient being processed with the vehicles, fillers, substances that influence decomposition, binding agents, moisturizing agents, lubricants, absorbing agents, diluents, flavoring correctives, coloring agents, etc., that are commonly used in galenicals and converted into the desired form of administration. In this case, reference is made to Remington's Pharmaceutical Science, 15^{th} Edition, Mack Publishing Company, East Pennsylvania (1980).

For oral administration, in particular tablets, coated tablets, capsules, pills, powders, granulates, lozenges, suspensions, emulsions or solutions are suitable. For parenteral administration, injection and infusion preparations are possible. For intraarticular injection, correspondingly prepared crystal suspensions can be used.

For intramuscular injection, aqueous and oily injection solutions or suspensions and corresponding depot preparations can be used.

For rectal administration, the new compounds can be used in the form of suppositories, capsules, solutions (e.g., in the form of enemas) and ointments both for systemic and for local therapy.

For pulmonary administration of the new compounds, the latter can be used in the form of aerosols and inhalants.

For topical application, formulations in gels, ointments, fatty ointments, creams, pastes, powders, milks and tinctures are possible. The dosage of the compounds of general formula I should be 0.01%-20% in these preparations to achieve an adequate pharmacological action.

This invention comprises the use of the compounds of general formula I according to the invention for the production of a pharmaceutical agent, in particular for treating tumor diseases that can be influenced positively by the inhibition of tubulin polymerization.

The compounds of general formula I according to the invention are preferably used for the production of a pharmaceutical agent, in particular for treating tumor diseases of the male and female gonads, male and female sex organs including the mammary glands, in particular of prostate cancer or breast cancer.

This invention also relates to pharmaceutical compositions that contain at least one especially preferred compound according to the invention, optionally in the form of a pharmaceutically/pharmacologically compatible salt, without or together with pharmaceutically compatible adjuvants and/or vehicles.

These pharmaceutical compositions and pharmaceutical agents can be provided for oral, rectal, vaginal, subcutaneous, percutaneous, intravenous or intramuscular administration. In addition to commonly used vehicles and/or diluents, they contain at least one especially preferred compound according to the invention.

The pharmaceutical agents of the invention are produced with commonly used solid or liquid vehicles or diluents and the commonly used pharmaceutical-technical adjuvants corresponding to the desired type of administration at a suitable dosage in a known way. The preferred preparations consist in a dispensing form that is suitable for oral administration. Such dispensing forms are, for example, tablets, film tablets, coated tablets, capsules, pills, powders, solutions or suspensions or else depot forms.

The pharmaceutical compositions that contain at least one of the compounds according to the invention are preferably administered orally.

Parenteral preparations such as injection solutions are also considered. In addition, for example, suppositories and agents for vaginal application can also be mentioned as preparations.

Corresponding tablets can be obtained by, for example, mixing active ingredient with known adjuvants, for example inert diluents such as dextrose, sugar, sorbitol, mannitol, polyvinyl pyrrolidone, explosives such as corn starch or alginic acid, binding agents such as starch or gelatin, lubricants such as magnesium stearate or talc and/or agents for achieving a depot effect such as carboxylpolymethylene, carboxylmethyl cellulose, cellulose acetate phthalate or polyvinyl acetate. The tablets can also consist of several layers.

Coated tablets accordingly can be produced by coating cores, which are produced analogously to the tablets, with agents that are commonly used in tablet coatings, for example, polyvinyl pyrrolidone or shellac, gum Arabic, talc, titanium oxide, or sugar. In this case, the shell of the coated tablets can also consist of several layers, whereby the adjuvants that are mentioned above in the tablets can be used.

Solutions or suspensions with the compounds of general formula I according to the invention can contain additional taste-improving agents such as saccharine, cyclamate or sugar, as well as, e.g., flavoring substances such as vanilla or orange extract. In addition, they can contain suspending adjuvants such as sodium carboxy methyl cellulose or preservatives such as p-hydroxybenzoates.

Capsules that contain the compounds of general formula I can be produced, for example, by the compound(s) of general formula I being mixed with an inert vehicle such as lactose or sorbitol and encapsulated in gelatin capsules.

Suitable suppositories can be produced by, for example, mixing with vehicles that are provided for this purpose, such as neutral fats or polyethylene glycol or derivatives thereof.

For therapy of prostate cancer, the compounds according to the invention can be administered in combination with one or more of the following active ingredients:

1) Antiandrogens such as CPA, flutamide, casodex, etc.

2) Gonadotrophic hormone (GnRH) agonists

3) 5 α-Reductase inhibitors such as finasteride

4) Cytostatic agents

5) VEGF-kinase inhibitors

6) Antigestagens

7) Antiestrogens

8) Antisense oligonucleotides

9) EGF antibodies

10) Estrogens

Moreover, the compounds of general formula I according to the invention can be used for therapy and prophylaxis of other pathologic conditions that are not mentioned above.

The invention also comprises the production of compounds according to general formula I.

The compounds of general formula I according to the invention can be produced as described below:

General Synthesis Part

The functionalization of C-atom 2 of an estra-1,3,5(10)-trien-17-one derivative is preferably carried out by Friedel-Crafts acylation as described in the literature (T. Nambara et al. Chem. Pharm. Bull. 1979, 18, 474-480).

After changing the protective group in 3-position, a 2-carboxy-estra-1,3,5(10)-trien-17-one is generated by Baeyer-Villiger oxidation (M. B. Smith, J. March, March's Advanced Organic Chemistry, 5^{th} Edition, Wiley Sons 2001, 1417-1418 and literature cited there). The ester is saponified and converted with the corresponding alkyl halide under basic conditions into a 2-alkyl ether. Alternately, the 17-ketone as known can now be reduced and etherified. The cleavage of the protective group in 3-position is carried out as described in the literature (T. W. Greene, P. G. M. Wuts, Protective Groups in Organic Synthesis, Wiley & Sons, 1999, 249-275). This process or other processes known from the literature (P. N. Rao, J. W. Cessac, Steroids 2002, 67, 1065-1070 and literature cited there) can be used according to the 18a-homo derivatives.

The 2-acyl derivatives that are preferably obtained by Friedel-Crafts acylation can be converted by reduction with sodium borohydride and subsequent hydrogenation into the corresponding 2-alkyl derivatives.

The 2-hydroxylation, starting from compounds of general formula II (R₂═H), 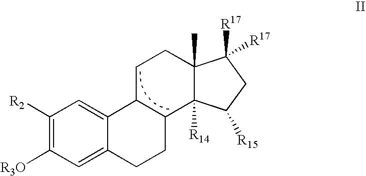
in which R₁₄ and R₁₅ together form a methylene bridge or which have additional double bonds in the steroid skeleton, is produced by ortho-metallation, whereby preferably an ether protective group (e.g., H. E. Paaren, S. R. Duff, U.S. Pat. No. 6,448,419 and literature cited there) or carbamate protective group (V. Snieckus, Chem. Rev. 1990, 90, 879-933) is used for R₃ as an ortho-directing protective group. The electrophilic substitution is carried out after 2-lithiation with trialkyl borate and subsequent basic oxidation with hydrogen peroxide. The selectively obtained 2-hydroxy group can then be converted in a known way (Z. Wang, M. Cushman, Synth. Commun. 1998, 28, 4431) into the 2-alkoxy compound and deprived of protection. Subsequent Oppenauer oxidation (C. Djerassi, Org. React. 1951, 6, 207, S. Schwarz et al. Pharmazie 2001, 56, 843-849) yields the 17-keto compounds, which can be further functionalized and reacted, as known, to form the sulfamates.

Starting from the 2-functionalized 17-keto derivatives, the 17-keto function can be removed by a Wolff-Kishner reduction (e.g., R. H. Peters et al., J. Med. Chem. 1989, 32, 1642; G. E. Agoston et al. WO 02/42319) and then sulfamoylated in 3-position.

This invention is explained in more detail based on the examples below, without being limited thereto:

### Production Process

General Synthesis Instructions 1 for the Production of Sulfamates

One equivalent of an estra-1,3,5(10)-triene derivative in methylene chloride is dissolved or suspended while being stirred and mixed with 5 equivalents of 2,6-di-tert-butylpyridine. Then, 10 equivalents of sulfamoyl chloride are added under argon and stirred at room temperature. The solution is stirred until conversion is completed (TLC monitoring, 1-5 hours) and then mixed with water. In acid-sensitive compounds, buffering is done in advance with about 10 equivalents of triethylamine. The aqueous phase is extracted several times with dichloromethane or ethyl acetate. The combined organic phases are dried on sodium sulfate and concentrated by evaporation in a vacuum and then purified by flash chromatography.

General Synthesis Instructions 2 for Acylation of Sulfamates

One equivalent of estra-1,3,5(10)-triene-sulfamate or bissulfamate is dissolved in pyridine and mixed with 5 equivalents of anhydride while being cooled with ice (0 to 5° C.). Stirring is continued for 1 hour at room temperature and then mixed with water. The aqueous phase is extracted several times with dichloromethane or ethyl acetate. The combined organic phases are washed with 6N hydrochloric acid and then with water and sodium chloride solution. Then, it is dried on sodium sulfate and concentrated by evaporation in a vacuum and then purified by flash chromatography.

### EXAMPLE 1

### 17β-Hydroxy-2-methoxy-18a-homoestra-1,3,5(10)-trien-3-yl sulfamate (1)

400 mg of 2-methoxy-17-oxo-18a-homoestra-1,3,5(10)-trien-3-yl sulfamate was dissolved in 5 ml of methanol and 5 ml of tetrahydrofuran and mixed at room temperature with two spatula-tips full of sodium borohydride. After 2 hours, it was acidified with 1N hydrochloric acid and concentrated by evaporation in a rotary evaporator. The residue was mixed with water and extracted with ethyl acetate (3x). The combined organic phases were washed with saturated sodium chloride solution, dried and concentrated by evaporation in a rotary evaporator. 425 mg (quant.) of 17β-hydroxy-2-methoxy-18a-homoestra-1,3,5(10)-trien-3-yl sulfamate (1) was obtained as a colorless foam.

¹H-NMR (DMSO-d₆): δ=0.93 (t, J=7.4 Hz, 3H; 18-CH₃), 2.70-2.74 (m, 2H; 6-CH₂), 3.60 (m, 1H; 17α-H), 3.76 (s, 3H; 2-OCH₃), 4.54 (d, J=4.3 Hz, 1H; OH), 6.96(s, 2H; 1-H, 4-H), 7.79 (br s, 2H; NH₂).

### EXAMPLE 2

### 2-Ethoxy-17β-hydroxy-18a-homoestra-1,3,5(10)trien-3-yl sulfamate (2)

400 mg of 2-ethoxy-17-oxo-18a-homoestra-1,3,5(10)-trien-3-yl sulfamate was dissolved in 5 ml of methanol and 5 ml of tetrahydrofuran and mixed at room temperature with two spatula-tips full of sodium borohydride. After 2 hours, it was acidified with 1N hydrochloric acid and concentrated by evaporation in a rotary evaporator. The residue was mixed with water and extracted with ethyl acetate (3x). The combined organic phases were washed with saturated sodium chloride solution, dried and concentrated by evaporation in a rotary evaporator. 387 mg (96%) of 2-ethoxy-17β-hydroxy-18a-homoestra-1,3,5(10)-trien-3-yl sulfamate (Z) is obtained as a colorless foam.

¹H-NMR (DMSO-d₆): δ=0.93 (t, J=7.2 Hz, 3H; 18-CH₃), 1.30 (t, J=7.0 Hz, 3H; 2-CH₃), 2.71-2.73 (m, 2H; 6-CH₂), 3.59-3.62 (m, 1H; 17α-H), 4.01-4.06 (m, 2H; 2-OCH₂), 4.53 (d, J=4.3 Hz, 1H; OH), 6.95, 6.96 (2s, 2H; 1-H, 4-H), 7.76 (br s, 2H); NH₂).

### EXAMPLE 3

### 2-Ethoxy-18a-homoestra-1,3,5(10)-triene-3,17β-diyl bissulfamate (3)

144 mg of 2,17β-dihyroxy-2-ethoxy-18a-homoestra-1,3,5(10)-triene was reacted to form the product according to the general synthesis instructions 1 and then purified by flash chromatography (toluene/ethyl acetate=3:1). 125 mg (59%) of 2-ethoxy-18a-homoestra-1,3,5(10)-triene-3,17β-diyl bissulfamate (2) was obtained as an amorphous solid.

¹H-NMR (DMSO-d₆): δ=0.92 (t, J=7.0 Hz, 3H; 18-CH₃), 2.71-2.76 (m, 2H; 6-CH₂), 4.04 (q, 2H; 2-OCH₂), 4.36 (t, ³J=8.0 Hz, 1H; 17aα-H), 6.96, 6.97 (2 s, 2H; 1-H, 4-H), 7.39 (s, 2H; NH₂), 7.79 (s, 2H; NH₂).

### EXAMPLE 4

### 2-Methoxy-18a-homoestra-1,3,5(10)-trien-3-yl sulfamate (4)

3-Hydroxy-2-methoxy-18a-homoestra-1,3,5(10)-triene was reacted to form the product according to general synthesis instructions 1 and then purified by flash chromatography (toluene/ethyl acetate=20:1→10:1). 2-Methoxy-18a-homoestra-1,3,5(10)-trien-3-yl sulfamate (4) in 91% yield was obtained as colorless crystals.

¹H-NMR (CDCl₃): δ=0.81 (t, ³J=7.4 Hz, 3H; 18-CH₃), 2.78-2.81 (m, 2H; 6-CH₂), 3.86 (s, 3H; 2-OCH₃), 5.02 (s, 2H; NH₂), 6.92, 7.02 (2 s, 2H; 1-H, 4-H).

### EXAMPLE 5

### 2,17β-Dimethoxy-18a-homoestra-1,3,5(10)-trien-3-yl Sulfamate

¹7.00 (s, 1H), 6.90 (s, 1H), 5.14 (s, 2H), 3.86 (s, 3H), 3.36 (m, 4H), 2.78 (m, 2H), 2.44 (m, 1H), 2.20 (m, 2H), 2.08 (m, 1H), 1.87 (m, 1H), 1.62 (m, 2H), 1.5-1.1 (m, 8H), 0.98 (m, 3H)

### EXAMPLE 6

### 2-Ethyl-18a-homoestra-1,3,5(10)-triene-3,17β-diyl Sulfamate

¹7.92 (s, 2H), 7.38 (s, 2H), 7.20 (s, 1H), 7.00 (s, 1H), 4.36 (s, 1H), 2.80 (m, 2.62 (m, 2H), 2.32 (m, 2H), 2.18 (m, 2H), 1.80 (m, 1H), 1.64 (m, 1H), 1.52-1.10 (m, 12H), 0.92 (m, 3H)

1. Use of 2-substituted 18a-homoestra-1,3,5(10)-trien-3-yl sulfamates of general formula I 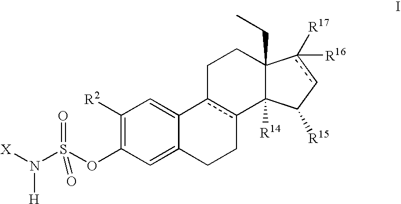
in which radicals R², R¹⁴ to R¹⁷ as well as X have the following meanings:
R² is C₁-C₅-alkyl, C₁-C₅-alkoxy or a radical —O—CₙFₘHₒ, whereby n=1, 2, 3, 4, 5 or 6, m>1 and m+o=2n+1,
R¹⁴ and R¹⁵ in each case are hydrogen or together a methylene group or an additional bond,
R¹⁶ is hydrogen,
R¹⁷ is hydrogen, hydroxy, C₁-C₅-alkyl, C₁-C₅-alkoxy or a radical —CₙFₘHₒ,
whereby n=1, 2, 3, 4, 5 or 6, m>1 and m+o=2n+1
or a group SO₃NHX,
X is hydrogen, C₁-C₅-alkyl or C₁-C₅-acyl,
whereby in the B-ring and D-ring of the steroid skeleton, the dotted lines can also be up to two double bonds, as well as their pharmaceutically acceptable salts for the production of a pharmaceutical agent.

2-19. (canceled)
